(19) United States
(12) Patent Application Publication
(10) Pub. No.: US 2018/0064598 A1
(43) Pub. Date: Mar. 8, 2018

(54) LOWER BACK PAIN TREATMENT AND SPINAL PROTECTION DEVICE

(71) Applicants: Rosalie Dudkiewicz, West Orange, NJ (US); Dariusz Dudkiewicz, West Orange, NJ (US)

(72) Inventors: Rosalie Dudkiewicz, West Orange, NJ (US); Dariusz Dudkiewicz, West Orange, NJ (US)

(21) Appl. No.: 15/807,525

(22) Filed: Nov. 8, 2017

Related U.S. Application Data
(63) Continuation-in-part of application No. 14/935,530, filed on Nov. 9, 2015.
Application No. 14/935,530 is a continuation-in-part of application No. 14/615,841, filed on Feb. 6, 2015, now Pat. No. 9,839,554.
(60) Provisional application No. 62/122,467, filed on Oct. 22, 2014.
(60) Provisional application No. 61/997,154, filed on May 27, 2014.

(51) Int. Cl. A61H 1/00; A61F 5/02

(52) U.S. Cl. CPC A61H 1/008; A61H 2201/1626; A61F 5/028; A61F 5/024

(57) ABSTRACT

A medical device is provided. The medical device includes a back panel, having a top surface and a bottom surface, and a plurality of pads attached to said top surface, wherein the plurality of pads includes an outer wall having an inner surface and an outer surface, and a rigid subwall proximate to said inner surface, wherein said medical device is configured to prevent incidental contact of a given object with a user's spinal column, and wherein said medical device prevents the compression of said user's spinal column.

### CLAIM OF PRIORITY

This application is a continuation-in-part of U.S. patent application Ser. No.14/935,530, filed on Nov. 9, 2015, entitled “LOWER BACK PAIN TREATMENT AND SPINAL PROTECTION DEVICE”, which is a continuation-in-part of U.S. patent application Ser. No. 14/615,841, filed on Feb. 6, 2015, entitled “LOWER BACK PAIN TREATMENT AND SPINAL PROTECTION DEVICE”, which claims priority from U.S. Provisional Patent Application No.: 62/122,467, filed on Oct. 22, 2014 and U.S. Provisional Patent Application No.: 61/997,154, filed on May 27, 2014 and the contents of which are hereby incorporated by reference.

### FIELD OF THE EMBODIMENTS

The invention and its embodiments relate to medical devices, namely a device that is capable of alleviating lower back pain. In particular, the present invention and its embodiments relate to a device that prevents the spinal column and its peripheral nerves from being touched or compressed.

### BACKGROUND OF THE EMBODIMENTS

There are about 150-200 million adults that suffer from low back pain in United States, and it is the second most common reason people seek a doctor. This health problem results in about 2.4 million Americans chronically disabled and temporarily on disability. Lower back pain affects the spine's stability, flexibility, and strength as result cause pain, discomfort and stiffness during daily living activities such as sitting, driving or sleeping. A great majority of back pain experienced by the general population occurs in the lower portion of the back generally referred to as the lumbar area or the spinal segments L-3 through S-1 specifically. In order to comprehend the most causes of low back pain, it is essential to understand the functioning of the spinal column. The purpose of the human spinal column is to support and protects the body. The most common area of low back pain is at the spinal process segment L-3 to S-1. The spinal column is composed of twenty four individual vertebrae. Each vertebra is attached and support by cartilages, muscles and ligaments to allow flexibility to bend over, and to rotate the torso. Each vertebra is connected by an intervertebral disc that functioning to provide cushion, and prevent compression to the peripheral spinal nerves. Whenever there is a shift of the vertebrae from its normal position caused by herniated discs, spinal stenosis, fractures, osteoarthritis or injury, it presses against the spinal nerves, and as a result it stimulates pain during daily living activities such as a sitting, sleeping, driving or bending. Likewise, low pain back is also caused by, the shifting of the 2 vertebrae caused by unequal pressure of the muscles supporting the spine column. This can occur due to overuse or injury of the muscles or ligaments, uneven muscular stress, emotional tension. The majority of low back pain is managed with conservative treatment such as rest, analgesic, anti-inflammatory medications in conjunction with physical therapy to strength the back muscles to support the spine column to relief pain or eliminate it. Another conventional treatment used to address back pain due to injury or non-injury is applying pressure to the area with orthopedic belt devices.

Review of Related Technology:

U.S. Pat. No. 8,226,587 pertains to a spinal support and brace device configured to support the lower back and that anchors on portion for the pelvis and are configured to support and align one or more lumber vertebrae.

International Patent Application No.: WO2007027573 pertains to a lumbar lordosis correction brace comprised of a lower portion, adapted to be comfortably affixed to the lower truck of a patient, and upper portion, adapted for comfortably encircling the upper trunk of a patient such that the two portions work together to provide uniform pressure against a patient's spine. This patent application also provides for the ability to displace, in a controlled manner, to provide additional support of the lumbar.

U.S. Pat. No. 5,405,313 pertains to an adjustable back support comprising a pelvic girdle that has been adapted to fit snugly around a user's waist, a pair of hip crutches adapted to be vertically disposed on each side of a user's body, wherein each hip crutch extends upwardly at a small forward angle between the pelvic girdle and the corresponding armpit.

US Patent Publication No.: 2010/0168630 pertains to a rigid lumbar compression piece, that includes first and second, separate, opposing and mating front attachment panels; and a separate, laterally adjustable, rigid lumbar compression piece configured for positioning only at the rear of a wearer, wherein pulling of a cord causes the brace both circumferentially to tighten and to concentrate compression and pressure of the separate, laterally adjustable, rigid lumbar compression piece pushed directly against a spinal region of a wearer's back with the aid of a number of apertures through which the cord or cords pass.

US Patent Publication No.: 2013/0090585 pertains to a neuromuscular training device for enhancing the alignment of the upper torso of a user. The device is preferably configured specifically to provide scapula retraction and stabilization while providing stated and dynamic neuromuscular training.

U.S. Pat. No. 5,334,134 pertains to a lumbosacral back support member which is releasably secured to the user by a stabilizing belt. Generally, this device is used over a user's clothes. This stabilizing belt may be inserted into the belt loops of a user's pants, attached to these belt loops, or engaged within the narrow portions of the user's waist.

U.S. Pat. No. 5,399,150 pertains to a lumbosacral back support system with interchangeably and positionally-adjustable lumbosacral orthopedic support members for supporting the spine of a user during physical activity, athletics activities, or for use by industrial workers.

U.S. Pat. No. 8,012,113 pertains to a spinal brace that includes a flexible air injectable band configured to be disposed about a torso of a user and to provide traction to a spine of the user. The device also comprises at least one support panel configured to provide support by compression to at least one region of a user's torso.

US Patent Publication No.: 2014/0221892 pertains to a maternity support belt that includes a curved support edge which matches the female pelvic curve, to support a gravid abdomen. The maternity belt includes a lumbosacral supporting section that applies a compressive force against the lumbosacral portion of the wearer's back, and a pair of gravid abdominal support sections on opposite lateral sides of the lumbosacral supporting section and contiguous thereto. The gravid abdominal support sections adjustably overlap and connect to each other on the wearer's front side.

U.S. Design Pat. No.: D657063 pertains to a back support.

Various devices are known in the art. However, their structure and means of operation are substantially different from the present invention. Such devices fail to provide a device that can be easily operated through medical gloves and that provide a tool that can be used on a wider array of hemorrhoidal tissue. At least one embodiment of this invention is presented in the drawings below and will be described in more detail herein.

### SUMMARY OF THE EMBODIMENTS

According to an aspect of the present invention, a medical device is provided. The medical device includes a back panel, having a top surface and a bottom surface, and a plurality of pads attached to said top surface, said plurality of pads comprising an outer wall having an inner surface and an outer surface, and a rigid subwall proximate to said inner surface, wherein said medical device is configured to prevent incidental contact of a given object with a user's spinal column, and wherein said medical device prevents the compression of said user's spinal column.

It is an object of the present invention to provide the medical device, wherein the plurality of pads further includes an expandable material located between said outer wall and said top surface.

It is an object of the present invention to provide the medical device, wherein said back panel is substantially rigid.

It is an object of the present invention to provide the medical device, wherein said medical device is configured to promote proper posture by properly aligning a user's spine.

It is an object of the present invention to provide the medical device, wherein said plurality of pads is equipped with a hollow center portion, sized to accommodate a growth of a cyst or tumor of a user proximate to said plurality of pads.

It is an object of the present invention to provide the medical device, wherein said plurality of pads are semi-cylindrical.

It is an object of the present invention to provide the medical device, wherein said plurality of pads are configured to be disposed on a plurality of areas of said top surface.

It is an object of the present invention to provide the medical device, wherein said plurality of pads is removably detached to said top surface.

It is an object of the present invention to provide the medical device, wherein said plurality of pads are removably attached to said surface via a connection mechanism.

It is an object of the present invention to provide the medical device, wherein said connection mechanism includes a plurality of hook and loop fasteners.

It is an object of the present invention to provide the medical device, wherein said medical device is constructed out of a material that is imbued with an antimicrobial agent.

It is an object of the present invention to provide the medical device, wherein said antimicrobial agent includes silver nanoparticles.

It is an object of the present invention to provide the medical device, wherein the plurality of pads are separated by a cavity configured to accommodate the spinal column.

According to another aspect of the present invention, a device for promoting proper curvature of a user's spinal column is provided. The device includes a back panel, having a top surface and a bottom surface, and a plurality of pads attached to said top surface of the back panel, said plurality of pads comprising an outer wall having an inner surface and an outer surface, a rigid subwall proximate to said inner surface, wherein each of the plurality of pads includes a concave cavity formed along a center portion, wherein said medical device is configured to prevent incidental contact of a given object with a user's spine, and wherein said medical device is configured to prevent compression of said user's spinal column.

It is an object of the present invention to provide the device for promoting proper curvature of the user's spinal column, wherein the concave cavity is configured to accommodate vertical and horizontal curves of lower back muscles of the user.

### BRIEF DESCRIPTION OF THE DRAWINGS

FIG. 1 is a top perspective view of an embodiment of the present invention.

FIG. 2a is a left side view of an embodiment of the present invention.

FIG. 2b is a right side view of an embodiment of the present invention.

FIG. 3 is a bottom view of an embodiment of the present invention.

FIG. 4 is an alternative top view of an embodiment of the present invention, with the pads of the present invention being removed.

FIG. 5 is a rear perspective view of an embodiment of the pads of the present invention.

FIG. 6 is a perspective view of an alternative embodiment of the pads of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

Low Back Pain solution device features two main components: the outside envelope and the back section. These components can be constructed from, by non-limiting example ethyl vinyl acetate (EVA) or nitrile butadiene rubber foam. In one embodiment, the back section is a single piece shaped similarly to a human buttock. In a preferred embodiment, the back section features two cushion pads, as well as a cavity. Preferably, this cavity will be 7 centimeters wide. The purpose of this cavity is to protect the spinal column and peripheral nerves from being touched or compressed by any object. Objects that can frequently cause such touching or compression include chairs, sofas, beds or car seats. This function also prevents pressure against incision site of post-spine surgery individual or individual with injured back. It allows the individual to perform daily activities such as sleeping, sitting or driving.

Further, any spinal incision is protected from being touched by any object during the above activities, which makes a user's healing process fast and effective. In turn, this makes the recovery period shorter. Another significant feature of this device is the two soft yet firm cushioned pads. Preferably, these pads have rectangular base with a top concave cavity 13 formed along a center portion of the pads, however, this does not mean that other shapes would not be suitable. The concave cavity 13 lies on the iliocostalis lumborum and quadratus lumborum muscles allowing sufficient space between the pads to accommodate the spinal column and its nerves to be free from contact due to any object. In a preferred embodiment, these pads 11 are aligned parallel to a user's lower spinal column and erector spinae musculature. These pads 11 hold the muscles in place to support the spine; allowing good blood flow to the area, and it prevents the muscles to fatigue, which also promotes healing.

The present invention also facilitates the correction of curvature and extension of a user's spinal column. This facilitation results in easing a user's back pain while helping them maintain proper posture. Additionally, the outside part has the purpose to enclose the back piece, and the front is made of spandex material that helps support the abdominal muscles, resulting in the promotion of proper posture and the supporting of the spine. In yet another preferred embodiment, the cavity has preferred dimensions of 7 cm wide while being and 2.5 cm deep, having a layer of 2.4 mm sheet of EVA and NBR foam. In one embodiment, the back section is enclosed by a comfortable non-spandex and washable fabric, but this should not be construed as the invention being limited to these fabrics.

According to an embodiment, the concave cavity 13 of the pads is configured to evenly distribute pressure on and de-pressure the back muscles, while the cavity between the pads 11 prevents any object from touching and/or compressing the spinal column (vertebrae bones, ligaments, disks, facets joints, spinal cord and nerves) while promoting proper posture by properly aligning a user's spine. According to an embodiment, the concave cavity pad 13 accommodates the vertical and horizontal curves of the lower back muscles without pressure points.

According to an embodiment, the cavity 13 is configured to evenly distribute pressure on and de-pressure the back muscles, while the cavity between the pads 11 prevents any object from touching and/or compressing the spinal column and nerves while promoting proper posture by properly aligning a user's spine. The concave cavity 13 accommodates the vertical and horizontal curves of the lower back muscles without pressure points.

According to an embodiment, the present invention provides a cavity 11 between the pads to shelter the spine (nerves, joints and disks) from been in contact with any object during,sitting, standing, or sleeping.

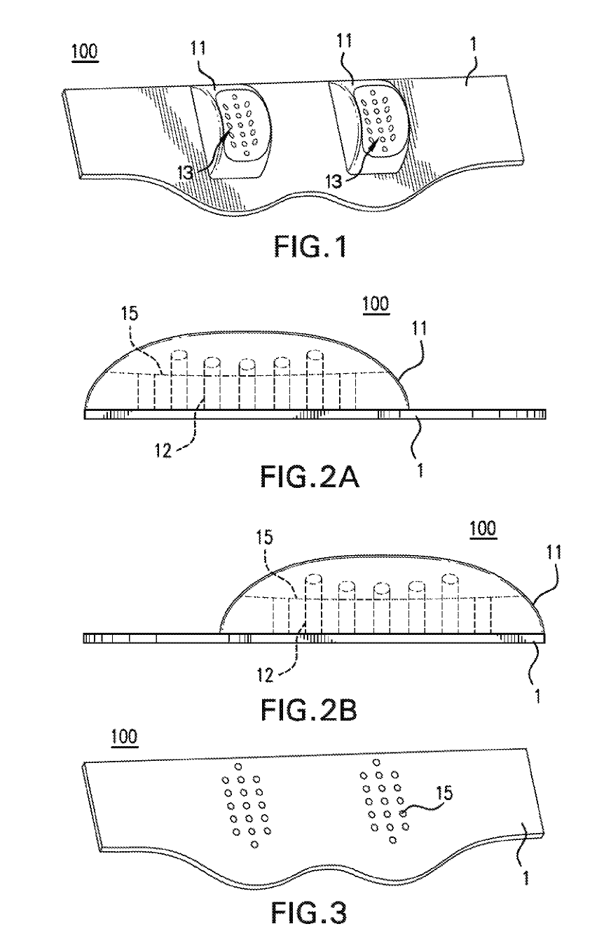

Referring now to FIG. 1, a perspective view of a medical device 100, according to an embodiment of the present invention, is shown. This embodiment features a back panel 1, one or more cavity pads 11, and a plurality of vents 12.

According to an embodiment, each of the one or more pads 11 has a semi-cylindrical shape. It is noted, however, that other shapes are contemplated by the present invention, while maintaining the spirit of the present invention. The present invention operates by securing the device 100 to a user's lower back, with the user's spine being located in between the plurality of pads 11. According to an embodiment, each of the pads 11 includes a concave cavity 13 formed along a center portion.

Once the device 100 is secured to the user, the plurality of pads 11 make contact with the area flanking the user's spine, resulting in compression of that spinal area being made impossible due to the support provided by plurality of pads 11. Further, due to the preferred rigidity of back panel 1, should a user bump up against an object inadvertently, their spine will still be shielded by the present invention.

According to an embodiment, the device may include one or more vents 12, as many of the types of materials suitable for use in constructing the plurality of cavity pads 11 are plastics that do not breathe. As such, the inclusion of the plurality of vents 12 will prevent a user from overheating while using the device 100 of the present invention. According to an embodiment, the device 100 does not include vents 12.

Referring now to FIG. 2, a side view of the device 100 is illustratively depicted, in accordance with an embodiment of the present invention. The view of FIG. 2 shows the relationship between the plurality of vents 12, the second opening 15, and the plurality of pads 11.

Referring now to FIG. 3, a bottom view of an embodiment of the present invention is provided.

According to the embodiment shown in FIG. 3, a second opening 15 of the plurality of vents 12 (See FIG. 2) is shown. As evidenced by FIG. 3, the second opening 15 is on the bottom surface of the rigid back panel of the present invention. This allows air from the ambient environment to be used to cool a user of the device 100 of the present invention.

Referring now to FIG. 4, an alternative top view of an embodiment of the present invention, with the pads of the present invention being removed, is illustratively depicted.

According to an embodiment, the back panel 1 is disposed with a plurality of loop fasteners. According to other embodiments, a plurality of hook fasteners are disposed on the back panel 1 in lieu of the plurality of loop fasteners. Here, the plurality of loop fasteners provide for the removable attachment of an embodiment of the pad 11 equipped with the plurality of hook fasteners.

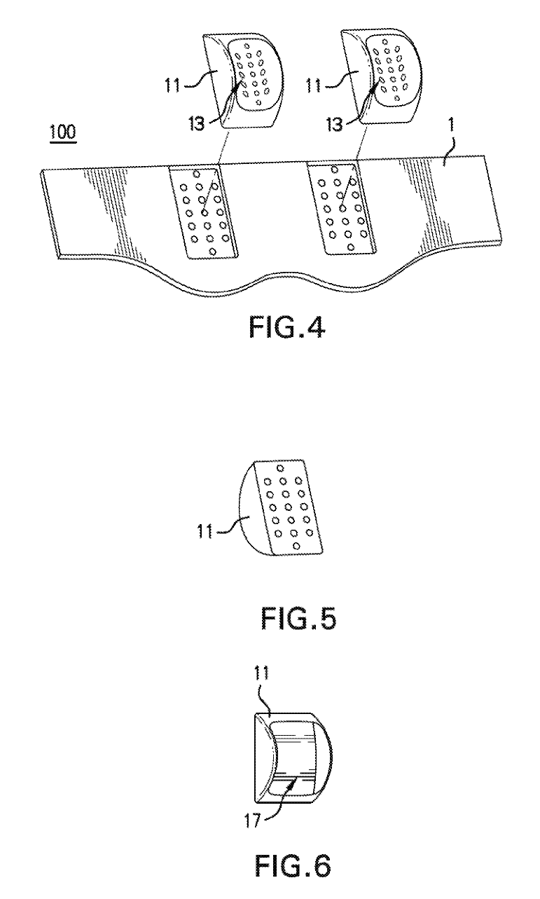

One benefit of this embodiment is that the device 100 may be more easily stored when the pads 11 (See FIG. 5) are detached from the back panel 1. Another benefit is that the user has the ability to adjust the distance between the pads 11 (See FIG. 5) when the pads 11 are attached to the back panel 1. This is beneficial because two or more given users may have spines with different widths, or may have other ailments that would result in more widely spaced pads 11 (See FIG. 5) being preferential.

Referring now to FIG. 5, a rear perspective view of an embodiment of the pads 11 of the present invention is illustratively depicted. This embodiment is intended to interface with the embodiment shown in FIG. 4. According to an embodiment, pad 11 is equipped with a plurality of hook fasteners. Much like in some other embodiments, the plurality of hook fasteners may be replaced with a plurality of loop fasteners. As noted above, this configuration provides for a more customizable experience while wearing the device 100 of the present invention.

Referring now to FIG. 6, a perspective view of an alternative embodiment of the pads of the present invention is illustratively depicted. This embodiment of the pads 11 features a hollow portion 17 in the center of the pad 11. According to some embodiments, pad 11 is removably attachable, but now a cavity 17 exists in the center of the pad 11. This cavity 17 is to accommodate a user who has grown a cyst, who has an implanted medical device, or who has some ether ailment that is sensitive to touch. This embodiment further enables the touch-sensitized area to be isolated, while still providing the back support and compression preventionthat the other embodiments of pads 11 provides, which is particularly useful for sleeping.

According to another embodiment of the present invention, the invention may be adapted and configured to support other bones of the body, including, but not limited to, supporting the bones of the arms and legs. According to another embodiment of the present invention, the pads 11 that create the channel or cavity into which the user's spine fits may be a unitary component. According to another embodiment, the present invention may be worn as an outer garment such as a corset. According to another embodiment, the present invention may be completely waterproof. According to another embodiment, the interior pads may be removable, adjustable and/or detachable. According to another embodiment of the invention, a user may insert the pads 11 into the device 100 and the pads 11 are customizable and may be placed to fit any user's spine or measurements. According to another embodiment, the pads 11 can be of any material and of various thicknesses to conform to a user's body. According to another embodiment, the present invention may contain heating or cooling pads within the pads 11 or within the channel or cavity 17.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. In some embodiments, the device of the present invention is constructed, in whole or in part out of a material containing an antimicrobial agent, such as silver nanoparticles.

While the disclosure refers to exemplary embodiments, it will be understood by those skilled in the art that various changes may be made and equivalents may be substituted for elements thereof without departing from the scope of the disclosure. In addition, many modifications will be appreciated by those skilled in the art to adapt a particular instrument, situation or material to the teachings of the disclosure without departing from the spirit thereof. Therefore, it is intended that the disclosure not be limited to the particular embodiments disclosed.

What is claimed is:

1. A medical device, comprising:
a back panel, having a top surface and a bottom surface; and
a plurality of pads attached to said top surface,
said plurality of pads comprising an outer wall having an inner surface and an outer surface, and a rigid subwall proximate to said inner surface,
wherein each of the plurality of pads includes a concave cavity formed along a center portion,
wherein said medical device is configured to prevent incidental contact of a given object with a user's spinal column, and
wherein said medical device prevents the compression of said user's spinal column.

2. The medical device as recited in claim 1, wherein the plurality of pads further includes an expandable material located between said outer wall and said top surface.

3. The medical device as recited in claim 1, wherein said back panel is substantially rigid.

4. The medical device as recited in claim 1, wherein said medical device is configured to promote proper posture by properly aligning a user's spine.

5. The medical device as recited in claim 1, wherein said plurality of pads is equipped with a hollow center portion, sized to accommodate a growth of a cyst or tumor of a user proximate to said plurality of pads.

6. The medical device as recited in claim 1, wherein said plurality of pads are semi-cylindrical.

7. The medical device as recited in claim 1, wherein said plurality of pads are configured to be disposed on a plurality of areas of said top surface.

8. The medical device as recited in claim 1, wherein said plurality of pads is removably detached from said top surface.

9. The medical device as recited in claim 1, wherein said plurality of pads are removably attached to said top surface via a connection mechanism.

10. The medical device as recited in claim 9, wherein said connection mechanism includes a plurality of hook and loop fasteners.

11. The medical device as recited in claim 1, wherein said medical device is constructed out of a material that is imbued with an antimicrobial agent.

12. The medical device as recited in claim 11, wherein said antimicrobial agent includes silver nanoparticles.

13. The medical device as recited in claim 1, wherein the plurality of pads are separated by a cavity configured to accommodate the spinal column.

14. A device for promoting a proper curvature of a user's spine, the device comprising:
a back panel, having a top surface and a bottom surface; and
a plurality of pads attached to said top surface,
said plurality of pads comprising an outer wall having an inner surface and an outer surface, and a rigid subwall proximate to said inner surface,
wherein each of the plurality of pads includes a concave cavity formed along a center portion,
wherein each of the plurality of pads include a plurality of vents,
wherein said medical device is configured to prevent incidental contact of a given object with a user's spinal column, and
wherein said medical device is configured to prevent compression of said user's spinal column.
